(19) United States
(12) Patent Application Publication
(10) Pub. No.: US 2025/0204924 A1
(43) Pub. Date: Jun. 26, 2025

(54) OCCLUSIVE IMPLANT SYSTEMS

(71) Applicant: BOSTON SCIENTIFIC SCIMED, INC., MAPLE GROVE, MN (US)

(72) Inventors: Patrick Willoughby, Shoreview, MN (US); David John Onushko, Maple Grove, MN (US)

(73) Assignee: BOSTON SCIENTIFIC SCIMED, INC., MAPLE GROVE, MN (US)

(21) Appl. No.: 18/987,233

(22) Filed: Dec. 19, 2024

Related U.S. Application Data
(60) Provisional application No. 63/612,493, filed on Dec. 20, 2023.
(60) Provisional application No. 63/612,507, filed on Dec. 20, 2023.
(60) Provisional application No. 63/612,569, filed on Dec. 20, 2023.
(60) Provisional application No. 63/612,582, filed on Dec. 20, 2023.
(60) Provisional application No. 63/561,406, filed on Mar. 5, 2024.
(60) Provisional application No. 63/561,415, filed on Mar. 5, 2024.
(60) Provisional application No. 63/560,160, filed on Mar. 1, 2024.
(60) Provisional application No. 63/560,174, filed on Mar. 1, 2024.

(51) Int. Cl. A61B 17/12; A61B 17/00; A61B 90/00

(52) U.S. Cl. CPC A61B 17/12172; A61B 17/12159; A61B 90/08; A61B 2017/00004; A61B 2017/00477; A61B 2017/1205; A61B 2017/12059; A61B 2017/12086; A61B 2017/12095; A61B 2090/0807

(57) ABSTRACT

An occlusive implant system may include a delivery catheter, a core wire including a connection element disposed at a distal end, and a piece of expandable foam coupled to the connection element. An occlusive implant system may include a delivery catheter, a core wire may include a clamshell enclosure disposed at a distal end, and a piece of expandable foam, wherein the clamshell enclosure is configured to deliver and thereafter release the piece of expandable foam at a treatment site. An occlusive implant system may include a delivery catheter, a core wire including a connection element disposed at a distal end, a piece of expandable foam engaged with the connection element, and a polymer coating disposed on the piece of expandable foam, wherein the polymer coating is configured to degrade in vivo to release the piece of expandable foam from the connection element.

### CROSS-REFERENCE TO RELATED APPLICATIONS

This application claims the benefit of priority of U.S. Provisional Application No. 63/612,493 filed Dec. 20, 2023, 63/612,507, filed Dec. 20, 2023, 63/612,569, filed Dec. 20, 2023, 63/612,582, filed Dec. 20, 2023, 63/561,406, filed Mar. 5, 2024, 63/561,415, filed Mar. 5, 2024, 63/560,160, filed Mar. 1, 2024, and 63/560,174, filed Mar. 1, 2024, the entirety disclosure of which is hereby incorporated by reference.

### TECHNICAL FIELD

The disclosure relates generally to medical devices and more particularly to medical devices that are adapted for use in percutaneous medical procedures including implantation into the left atrial appendage (LAA) of a heart.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

In one example, an occlusive implant system may comprise a delivery catheter having a lumen extending therethrough, a core wire slidably disposed within the delivery catheter, wherein the core wire comprises a connection element disposed at a distal end of the core wire, and a piece of foam coupled to the connection element. The piece of foam may be configured to expand from a delivery configuration to an expanded configuration.

In addition or alternatively to any example disclosed herein, a portion of the connection element is disposed within the piece of foam in the delivery configuration.

In addition or alternatively to any example disclosed herein, the piece of foam is compressed onto the portion of the connection element disposed within the piece of expandable foam in the delivery configuration such that the connection element is prevented from disengaging from the piece of foam when the piece of foam is in the delivery configuration.

In addition or alternatively to any example disclosed herein, the connection element is a helical coil.

In addition or alternatively to any example disclosed herein, the connection element is configured to be unscrewed from the piece of foam when the piece of foam is in the expanded configuration.

In addition or alternatively to any example disclosed herein, the connection element is configured to straighten and pull out of the piece of foam when tension is applied to the connection element and the piece of foam is in the expanded configuration.

In addition or alternatively to any example disclosed herein, the connection element is a wedge.

In addition or alternatively to any example disclosed herein, the connection element is disposed entirely within the piece of foam in the delivery configuration.

In addition or alternatively to any example disclosed herein, expansion of the piece of foam to the expanded configuration releases the connection element from the piece of foam.

In addition or alternatively to any example disclosed herein, the occlusive implant system may comprise a shielding element disposed about the piece of foam in the delivery configuration.

In addition or alternatively to any example disclosed herein, the piece of foam is non-releasably coupled to a medical implant configured to occlude a left atrial appendage, the medical implant comprising an expandable framework.

In addition or alternatively to any example disclosed herein, the piece of foam forms an occlusive disk in the expanded configuration.

In addition or alternatively to any example disclosed herein, the piece of foam comprises a second connection element disposed therein, the second connection element being configured to selectively disengage from the connection element to release the piece of foam.

In addition or alternatively to any example disclosed herein, a magnetic force selectively couples the connection element to the second connection element.

In addition or alternatively to any example disclosed herein, tension applied to the connection element after the piece of foam has expanded to the expanded configuration causes the connection element to disengage from the second connection element.

In addition or alternatively to any example disclosed herein, an occlusive implant system may comprise a delivery catheter having a lumen extending therethrough, a core wire slidably disposed within the delivery catheter, wherein the core wire comprises a clamshell enclosure disposed at a distal end of the core wire, and a piece of foam configured to expand from a delivery configuration to an expanded configuration. The clamshell enclosure may be configured to deliver the piece of foam to a treatment site and thereafter release the piece of foam at the treatment site.

In addition or alternatively to any example disclosed herein, the piece of foam is disposed within the clamshell enclosure in the delivery configuration.

In addition or alternatively to any example disclosed herein, the clamshell enclosure is releasably coupled to the piece of foam in the delivery configuration.

In addition or alternatively to any example disclosed herein, the piece of foam comprises a marker element disposed at a proximal end of the piece of foam, and the clamshell enclosure is configured to releasably attach to the marker element to deliver the piece of foam to a treatment site and thereafter detach from the marker element to release the piece of foam at the treatment site.

In addition or alternatively to any example disclosed herein, an occlusive implant system may comprise a delivery catheter having a lumen extending therethrough, a core wire slidably disposed within the delivery catheter, wherein the core wire comprises a connection element disposed at a distal end of the core wire, a piece of foam engaged with the connection element, the piece of foam being configured to expand from a delivery configuration to an expanded configuration, and a polymer coating disposed on the piece of foam in the delivery configuration, wherein the polymer coating is configured to degrade in vivo to release the piece of foam from the connection element. The piece of foam may be configured to begin expanding from the delivery configuration to the expanded configuration before the polymer coating releases the piece of foam from the connection element.

The above summary of some embodiments, aspects, and/or examples is not intended to describe each embodiment or every implementation of the present disclosure. The figures and the detailed description more particularly exemplify aspects of these embodiments.

### BRIEF DESCRIPTION OF THE DRAWINGS

The disclosure may be more completely understood in consideration of the following detailed description in connection with the accompanying drawings, in which:

FIGS. 1-3 schematically illustrate selected aspects of an occlusive implant system;

FIG. 4 schematically illustrates selected aspects of an alternative configuration of the occlusive implant system of FIGS. 1-3;

FIGS. 5-7 schematically illustrate selected aspects of an occlusive implant system;

FIGS. 8-10 schematically illustrate selected aspects of an occlusive implant system;

FIG. 11 schematically illustrates selected aspects of an alternative configuration of the occlusive implant systems of FIGS. 5-10;

FIGS. 12-13 schematically illustrate selected aspects of an occlusive implant system;

FIGS. 14-15 schematically illustrate selected aspects of an occlusive implant system;

FIGS. 16-18 schematically illustrate selected aspects of an occlusive implant system;

FIGS. 19-20 schematically illustrate selected aspects of an occlusive implant system disposed within a left atrial appendage; and

FIGS. 21-23 schematically illustrate selected aspects of an occlusive implant system disposed within a left atrial appendage.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

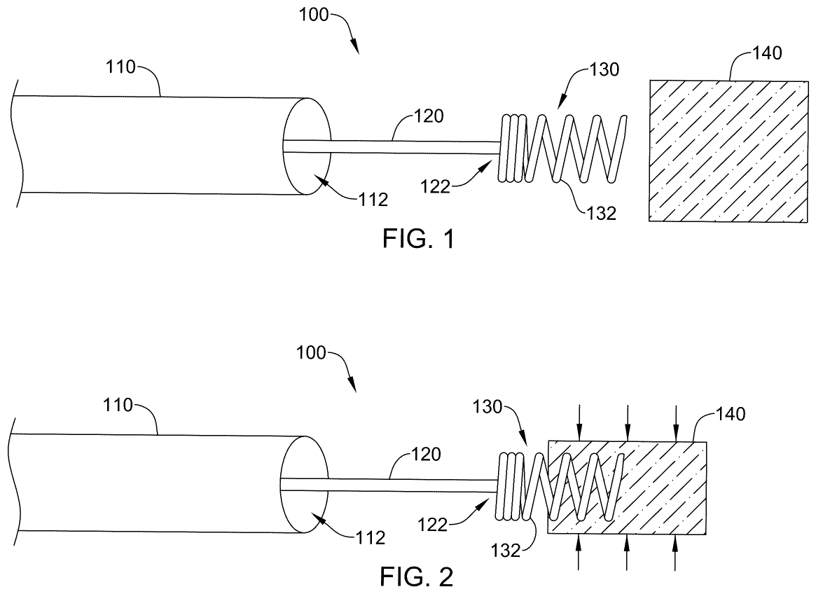

FIGS. 1-3 schematically illustrate selected aspects of an occlusive implant system 100. The occlusive implant system 100 may comprise a delivery catheter 110 having a lumen 112 extending therethrough. In some embodiments, the delivery catheter 110 may comprise a proximal hub and/or manifold (not shown). In some embodiments, the delivery catheter 110 may be formed from a tubular member. In some embodiments, the delivery catheter may comprise an additional lumen or lumens (e.g., a guidewire lumen, an aspiration lumen, a working lumen, etc.) extending therein and/or therethrough. Other configurations are also contemplated.

In some embodiments, the occlusive implant system 100 may comprise a core wire 120 slidably disposed within the delivery catheter 110 and/or the lumen 112 of the delivery catheter 110. In some embodiments, the core wire 120 may be formed with a solid cross-section. In some embodiments, the core wire 120 may be formed with a hollow cross-section (e.g., from a tubular member). In some embodiments, the core wire 120 may be formed from a metallic material. In some embodiments, the core wire 120 may be formed from a polymeric material. In some embodiments, the core wire 120 may be formed from a composite material.

The core wire 120 may comprise a connection element 130 disposed at a distal end 122 of the core wire 120. In at least some embodiments, the connection element 130 may be fixedly attached to the distal end 122 of the core wire 120. In some embodiments, the connection element 130 may be adhesively bonded, welded, etc. to the distal end 122 of the core wire 120. In some embodiments, the connection element 130 may be integrally formed with the core wire 120. In some embodiments, the connection element 130 may be monolithically formed with the core wire 120. Other configurations are also contemplated.

The occlusive implant system 100 may comprise a piece of expandable foam 140 coupled to the connection element 130. The piece of expandable foam 140 may be configured to expand from a delivery configuration (e.g., a compressed configuration) to an expanded configuration. While the piece of expandable foam 140 may be illustrated as having a particular shape, it shall be understood that the illustration is purely schematic and that other shapes and/or configurations are also contemplated within the scope of the disclosure. Additionally, it shall be understood that in some embodiments, the piece of expandable foam 140 may have a first shape in the delivery configuration and a second shape different from the first shape in the expanded configuration.

In at least some embodiments, the piece of expandable foam 140 may be coupled to the connection element 130 initially while the piece of expandable foam 140 is in the expanded configuration. Accordingly, in some embodiments, a portion of the connection element 130 may be disposed within the piece of expandable foam 140. Thereafter, the piece of expandable foam 140 may be compressed toward and/or to the delivery configuration such that the piece of expandable foam 140 is compressed onto the portion of the connection element 130 disposed within the piece of expandable foam 140 in the delivery configuration. In at least some embodiments, the connection element 130 may be prevented from disengaging from the piece of expandable foam 140 when the piece of expandable foam 140 is in the delivery configuration.

As discussed herein, the piece of expandable foam 140 may be configured to expand from the delivery configuration toward and/or to the expanded configuration in vivo (e.g., after deployment at the treatment site). In some embodiments, the piece of expandable foam 140 may be self-biased toward the expanded configuration. In some embodiments, the piece of expandable foam 140 may comprise a shape memory polymer and/or a shape memory foam. The shape memory polymer and/or the shape memory foam may have multiple geometric and/or mechanical properties when exposed to temperature, moisture, and/or chemical environments, and/or changes therein. In some embodiments, the shape memory polymer and/or the shape memory foam may have a collapsibility ratio that is high. The collapsibility ratio is a ratio between an expanded size and a collapsed size. In some examples, the collapsibility ratio of the shape memory polymer and/or the shape memory foam may be at least 5 times, at least 7 times, at least 8 times, at least 9 times, at least 10 times, at least 12 times, or more. In one example, the piece of expandable foam 140 may have an outer diameter of about 32 millimeters in the expanded configuration and about 3 millimeters in the compressed configuration, producing a collapsibility ratio of at least 10 times (e.g., at least 10:1). Other configurations are also contemplated. In at least some embodiments, the piece of expandable foam 140 may be configured as open celled foam.

In some embodiments, the piece of expandable foam 140 may be configured to shift from the delivery configuration toward and/or to the expanded configuration after being exposed and/or subjected to a trigger condition (e.g., a temperature change, an application or removal of an electrical current, a chemical change, exposure to fluid or a particular fluid type, etc.).

The piece of expandable foam 140 and/or the shape memory foam may be formed from a biocompatible material. In at least some embodiments, the piece of expandable foam 140 may be non-biodegradable and/or non-bioabsorbable. In some alternative embodiments, the piece of expandable foam 140 may be biodegradable and/or bioabsorbable over time. In some embodiments, the piece of expandable foam 140 may be configured to prevent thrombus formation. In some embodiments, the piece of expandable foam 140 may include anti-thrombus medicament(s). In some embodiments, the piece of expandable foam 140 may be configured to absorb blood and/or bodily fluid(s). In some embodiments, the piece of expandable foam 140 may be configured to trap thrombus. In some embodiments, the piece of expandable foam 140 may be configured to promote endothelization and/or tissue ingrowth. In some embodiments, the piece of expandable foam 140 may include a coating, a material, and/or a component that promotes endothelization and/or tissue ingrowth. Other configurations are also contemplated.

In some embodiments, the piece of expandable foam 140 may have a generally open structure (e.g., the piece of expandable foam 140 may have pores or open cells) in the expanded configuration. In some embodiments, the piece of expandable foam 140 may be configured to receive and/or accept the connection element 130 therein in the expanded configuration. Upon compression and/or crimping of the piece of expandable foam 140 onto the connection element 130, and/or toward and/or to the delivery configuration, the connection element 130 is or would be unable to disengage from, and/or is or would be prevented from, disengaging from the piece of expandable foam 140.

In some embodiments, the connection element 130 may comprise and/or may be a helical coil 132. In some embodiments, the helical coil 132 may be fixedly attached to the distal end 122 of the core wire 120. In some embodiments, the helical coil 132 may be integrally formed with the core wire 120 and/or monolithically formed with the core wire 120 such that the helical coil 132 is formed from the core wire 120 itself. Other configurations are also contemplated.

In some embodiments, the connection element 130 and/or the helical coil 132 may be configured to screw into the piece of expandable foam 140 and/or the generally open structure of the piece of expandable foam 140 in the expanded configuration. In some embodiments, the connection element 130 and/or the helical coil 132 may be configured to screw into the piece of expandable foam 140 and/or the generally open structure of the piece of expandable foam 140 via rotation of the core wire 120 in a first direction relative to the piece of expandable foam 140 in the expanded configuration. Thereafter, the piece of expandable foam 140 may be compressed and/or crimped onto the portion of the connection element 130 and/or the helical coil 132 that is disposed within the piece of expandable foam 140, as seen in FIG. 2. Once the piece of expandable foam 140 has been compressed and/or crimped onto the portion of the connection element 130 and/or the helical coil 132 that is disposed within the piece of expandable foam 140, the connection element 130 and/or the helical coil 132 may be prevented from unscrewing from and/or disengaging from the piece of expandable foam 140. In some embodiments, the portion of the connection element 130 and/or the helical coil 132 that is disposed within the piece of expandable foam 140 may only be permitted to unscrew and/or disengage from the piece of expandable foam 140 when the piece of expandable foam 140 is in the expanded configuration.

In some embodiments, compressing and/or crimping the piece of expandable foam 140 onto the portion of the connection element 130 and/or the helical coil 132 that is disposed within the piece of expandable foam 140 may change the density or other properties of the piece of expandable foam 140 such that the connection element 130 and/or the helical coil 132 may be prevented from unscrewing from and/or disengaging from the piece of expandable foam 140 when the piece of expandable foam 140 is in the delivery configuration. In some embodiments, compressing and/or crimping the piece of expandable foam 140 onto the portion of the connection element 130 and/or the helical coil 132 that is disposed within the piece of expandable foam 140 may cause and/or create mechanical interference between the piece of expandable foam 140 and the portion of the connection element 130 and/or the helical coil 132 that is disposed within the piece of expandable foam 140 such that the connection element 130 and/or the helical coil 132 may be prevented from unscrewing from and/or disengaging from the piece of expandable foam 140 when the piece of expandable foam 140 is in the delivery configuration.

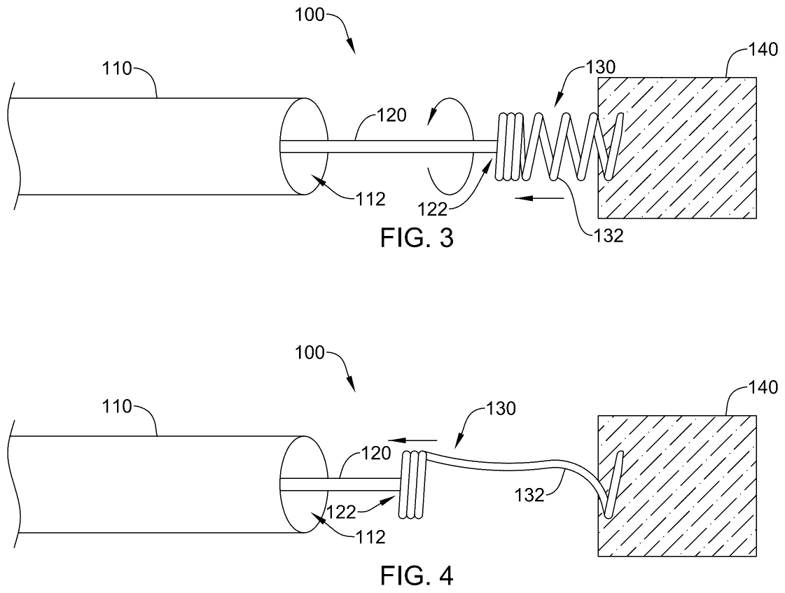

In some embodiments, after deploying the piece of expandable foam 140 at a treatment site, the piece of expandable foam 140 may be configured to expand from the delivery configuration to the expanded configuration. In some embodiments, the connection element 130 and/or the helical coil 132 may be configured to be unscrewed from the piece of expandable foam 140 when the piece of expandable foam 140 is in the expanded configuration, as seen in FIG. 3. In some embodiments, the connection element 130 and/or the helical coil 132 may be configured to be unscrewed from the piece of expandable foam 140 via rotation of the core wire 120 in a second direction opposite the first direction relative to the piece of expandable foam 140 when the piece of expandable foam 140 is in the expanded configuration.

In some embodiments, the connection element 130 and/or the helical coil 132 may be configured to straighten and pull out of the piece of expandable foam 140 when tension is applied to the connection element 130 and/or the helical coil 132 and the piece of expandable foam 140 is in the expanded configuration, as seen in FIG. 4. In some embodiments, the connection element 130 and/or the helical coil 132 may be constructed and/or may include properties and/or characteristics that permit the connection element 130 and/or the helical coil 132 to deform, elongate, and/or straighten under tension and/or when tension is applied thereto via the core wire 120, thereby permitting the connection element 130 and/or the helical coil 132 to be pulled from the piece of expandable foam 140 in the expanded configuration without significantly damaging the piece of expandable foam 140 and/or without compromising the structural integrity of the piece of expandable foam 140.

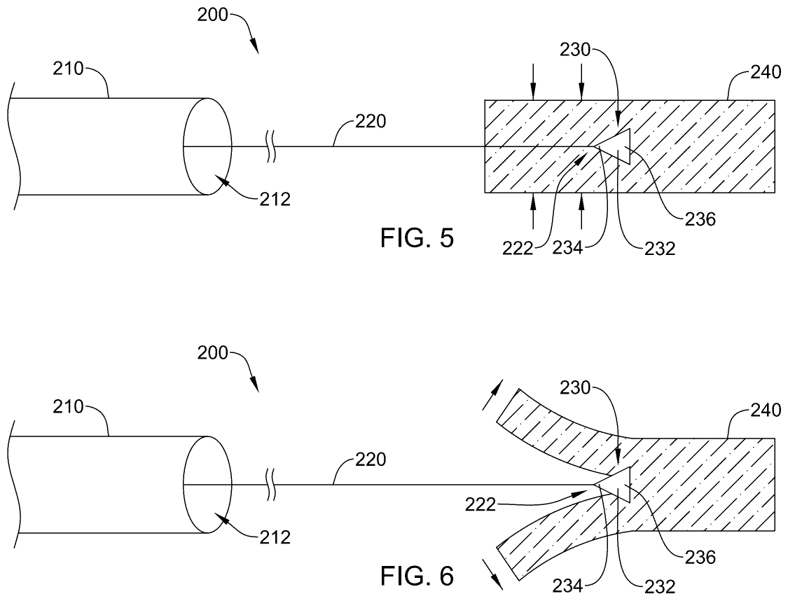

FIGS. 5-7 schematically illustrate selected aspects of an occlusive implant system 200. The occlusive implant system 200 may comprise a delivery catheter 210 having a lumen 212 extending therethrough. In some embodiments, the delivery catheter 210 may comprise a proximal hub and/or manifold (not shown). In some embodiments, the delivery catheter 210 may be formed from a tubular member. In some embodiments, the delivery catheter may comprise an additional lumen or lumens (e.g., a guidewire lumen, an aspiration lumen, a working lumen, etc.) extending therein and/or therethrough. Other configurations are also contemplated.

In some embodiments, the occlusive implant system 200 may comprise a core wire 220 slidably disposed within the delivery catheter 210 and/or the lumen 212 of the delivery catheter 210. In some embodiments, the core wire 220 may be formed with a solid cross-section. In some embodiments, the core wire 220 may be formed with a hollow cross-section (e.g., from a tubular member). In some embodiments, the core wire 220 may be formed from a metallic material. In some embodiments, the core wire 220 may be formed from a polymeric material. In some embodiments, the core wire 220 may be formed from a composite material.

The core wire 220 may comprise a connection element 230 disposed at a distal end 222 of the core wire 220. In at least some embodiments, the connection element 230 may be fixedly attached to the distal end 222 of the core wire 220. In some embodiments, the connection element 230 may be integrally formed with the core wire 220. In some embodiments, the connection element 230 may be monolithically formed with the core wire 220. Other configurations are also contemplated.

The occlusive implant system 200 may comprise a piece of expandable foam 240 coupled to the connection element 230. The piece of expandable foam 240 may be configured to expand from a delivery configuration (e.g., a compressed configuration) to an expanded configuration. While the piece of expandable foam 240 may be illustrated as having a particular shape, it shall be understood that the illustration is purely schematic and that other shapes and/or configurations are also contemplated within the scope of the disclosure. Additionally, it shall be understood that in some embodiments, the piece of expandable foam 240 may have a first shape in the delivery configuration and a second shape different from the first shape in the expanded configuration.

In at least some embodiments, the piece of expandable foam 240 may be coupled to the connection element 230 initially while the piece of expandable foam 240 is in the expanded configuration. Accordingly, in some embodiments, a portion of the connection element 230 may be disposed within the piece of expandable foam 240. Thereafter, the piece of expandable foam 240 may be compressed toward and/or to the delivery configuration such that the piece of expandable foam 240 is compressed onto the portion of the connection element 230 disposed within the piece of expandable foam 240 in the delivery configuration. In some embodiments, the connection element 230 may be disposed entirely within the piece of expandable foam 240 in the delivery configuration. In at least some embodiments, the connection element 230 may be prevented from disengaging from the piece of expandable foam 240 when the piece of expandable foam 240 is in the delivery configuration.

As discussed herein, the piece of expandable foam 240 may be configured to expand from the delivery configuration toward and/or to the expanded configuration in vivo (e.g., after deployment at the treatment site). In some embodiments, the piece of expandable foam 240 may be self-biased toward the expanded configuration. In some embodiments, the piece of expandable foam 240 may comprise a shape memory polymer and/or a shape memory foam. The shape memory polymer and/or the shape memory foam may have multiple geometric and/or mechanical properties when exposed to temperature, moisture, and/or chemical environments, and/or changes therein. In some embodiments, the shape memory polymer and/or the shape memory foam may have a collapsibility ratio that is high. The collapsibility ratio is a ratio between an expanded size and a collapsed size. In some examples, the collapsibility ratio of the shape memory polymer and/or the shape memory foam may be at least 5 times, at least 7 times, at least 8 times, at least 9 times, at least 10 times, at least 12 times, or more. In one example, the piece of expandable foam 240 may have an outer diameter of about 32 millimeters in the expanded configuration and about 3 millimeters in the compressed configuration, producing a collapsibility ratio of at least 10 times (e.g., at least 10:1). Other configurations are also contemplated. In at least some embodiments, the piece of expandable foam 240 may be configured as open celled foam.

In some embodiments, the piece of expandable foam 240 may be configured to shift from the delivery configuration toward and/or to the expanded configuration after being exposed and/or subjected to a trigger condition (e.g., a temperature change, an application or removal of an electrical current, a chemical change, etc.).

The piece of expandable foam 240 and/or the shape memory foam may be formed from a biocompatible material. In at least some embodiments, the piece of expandable foam 240 may be non-biodegradable and/or non-bioabsorbable. In some alternative embodiments, the piece of expandable foam 240 may be biodegradable and/or bioabsorbable over time. In some embodiments, the piece of expandable foam 240 may be configured to prevent thrombus formation. In some embodiments, the piece of expandable foam 240 may include anti-thrombus medicament(s). In some embodiments, the piece of expandable foam 240 may be configured to absorb blood and/or bodily fluid(s). In some embodiments, the piece of expandable foam 240 may be configured to trap thrombus. In some embodiments, the piece of expandable foam 240 may be configured to promote endothelization and/or tissue ingrowth. In some embodiments, the piece of expandable foam 240 may include a coating, a material, and/or a component that promotes endothelization and/or tissue ingrowth. Other configurations are also contemplated.

In some embodiments, the piece of expandable foam 240 may have a generally open structure (e.g., the piece of expandable foam 240 may have pores or open cells) in the expanded configuration. In some embodiments, the piece of expandable foam 240 may be configured to receive and/or accept the connection element 230 therein in the expanded configuration. Upon compression and/or crimping of the piece of expandable foam 240 onto the connection element 230, and/or toward and/or to the delivery configuration, the connection element 230 is or would be unable to disengage from, and/or is or would be prevented from, disengaging from the piece of expandable foam 240.

In some embodiments, the connection element 230 may comprise and/or may be a wedge 232. In some embodiments, the wedge 232 may be and/or may have a three-dimensional shape. In some embodiments, the three-dimensional shape may include a narrow end 234 and a wide end 236, wherein the narrow end 234 is configured to couple directly to the core wire 220 and the wide end 236 is disposed opposite the narrow end 234 and/or is spaced apart from the narrow end 234. As such, in some embodiments, the wedge 232 may have a pyramidal shape, a prismatic shape, a conical shape, etc. In some embodiments, the wedge 232 may be fixedly attached to the distal end 222 of the core wire 220. In some embodiments, the narrow end 234 of the wedge 232 may be fixedly attached directly to the distal end 222 of the core wire 220. In some embodiments, the wedge 232 may be integrally formed with the core wire 220 and/or monolithically formed with the core wire 220 with the wide end 236 of the wedge 232 disposed opposite and/or spaced apart from the distal end 222 of the core wire 220. Other configurations are also contemplated.

In some embodiments, a portion of the connection element 230 and/or the wedge 232, and/or an entirety of the connection element 230 and/or the wedge 232, may be inserted into the piece of expandable foam 240 and/or the generally open structure of the piece of expandable foam 240 in the expanded configuration. In some embodiments, the piece of expandable foam 240 may be formed around the portion of the connection element 230 and/or the wedge 232, and/or the entirety of the connection element 230 and/or the wedge 232. Thereafter, the piece of expandable foam 240 may be compressed and/or crimped onto the portion of the connection element 230 and/or the wedge 232 that is disposed within the piece of expandable foam 240, as seen in FIG. 5. Once the piece of expandable foam 240 has been compressed and/or crimped onto the portion of the connection element 230 and/or the wedge 232 that is disposed within the piece of expandable foam 240, the connection element 230 and/or the wedge 232 may be prevented from disengaging from and/or pulling out of the piece of expandable foam 240. In some embodiments, the connection element 230 and/or the wedge 232 that is disposed within the piece of expandable foam 240 may only be permitted to release and/or disengage from the piece of expandable foam 240 when the piece of expandable foam 240 is in the expanded configuration.

In some embodiments, compressing and/or crimping the piece of expandable foam 240 onto the portion of the connection element 230 and/or the wedge 232 that is disposed within the piece of expandable foam 240 may change the density or other properties of the piece of expandable foam 240 such that the connection element 230 and/or the wedge 232 may be prevented from releasing from and/or disengaging from the piece of expandable foam 240 when the piece of expandable foam 240 is in the delivery configuration. In some embodiments, compressing and/or crimping the piece of expandable foam 240 onto the portion of the connection element 230 and/or the wedge 232 that is disposed within the piece of expandable foam 240 may cause and/or create mechanical interference between the piece of expandable foam 240 and the portion of the connection element 230 and/or the wedge 232 that is disposed within the piece of expandable foam 240 such that the connection element 230 and/or the wedge 232 may be prevented from releasing from and/or disengaging from the piece of expandable foam 240 when the piece of expandable foam 240 is in the delivery configuration. In some embodiments, the piece of expandable foam 240 may have and/or may assume a first shape and/or a first form in the delivery configuration, and the piece of expandable foam 240 may have and/or may assume a second shape and/or a second form in the expanded configuration that is different from the first shape and/or the first form.

In some embodiments, after deploying the piece of expandable foam 240 at a treatment site, the piece of expandable foam 240 may be configured to expand from the delivery configuration toward and/or to the expanded configuration, as seen in FIG. 6. In some embodiments, after deploying the piece of expandable foam 240 at the treatment site, the piece of expandable foam 240 may be configured to release the connection element 230 in the expanded configuration. In some embodiments, after deploying the piece of expandable foam 240 at the treatment site, the piece of expandable foam 240 may be configured to change shape and/or assume a different form that permits the connection element 230 and/or the wedge 232 to release from and/or disengage from the piece of expandable foam 240.

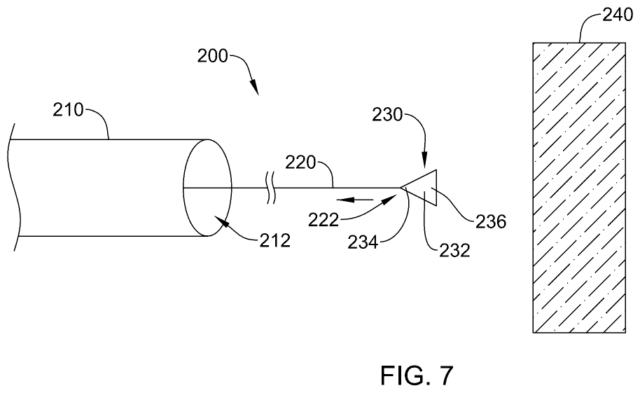

In some embodiments, expansion of the piece of expandable foam 240 toward and/or to the expanded configuration may release the connection element 230 and/or the wedge 232 from the piece of expandable foam 240 such that the connection element 230 and/or the wedge 232 may disengage from the piece of expandable foam 240, as seen in FIG. 7. In some embodiments, the connection element 230 and/or the wedge 232 may be configured to pull out of the piece of expandable foam 240 when tension is applied to the connection element 230 and/or the wedge 232 and the piece of expandable foam 240 is in the expanded configuration. Other configurations are also contemplated. In at least some embodiments, the piece of expandable foam 240 may form an occlusive disk in the expanded configuration.

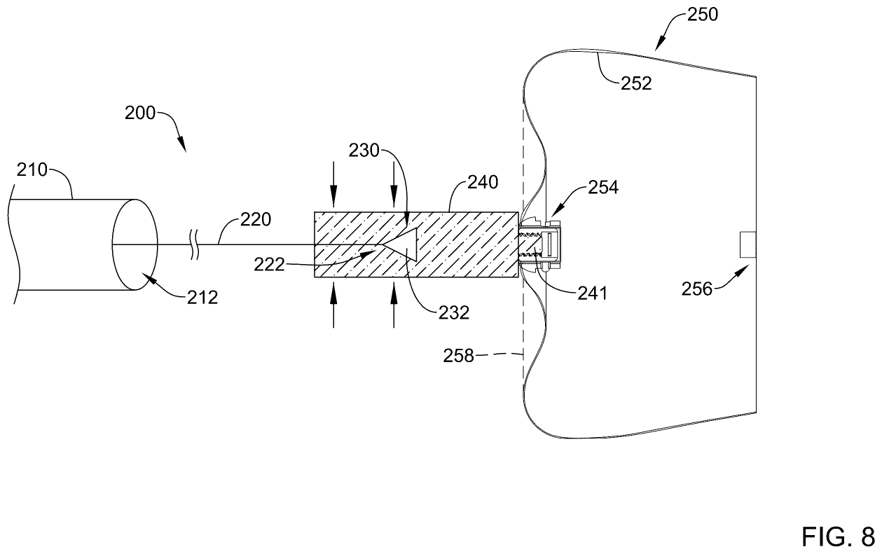

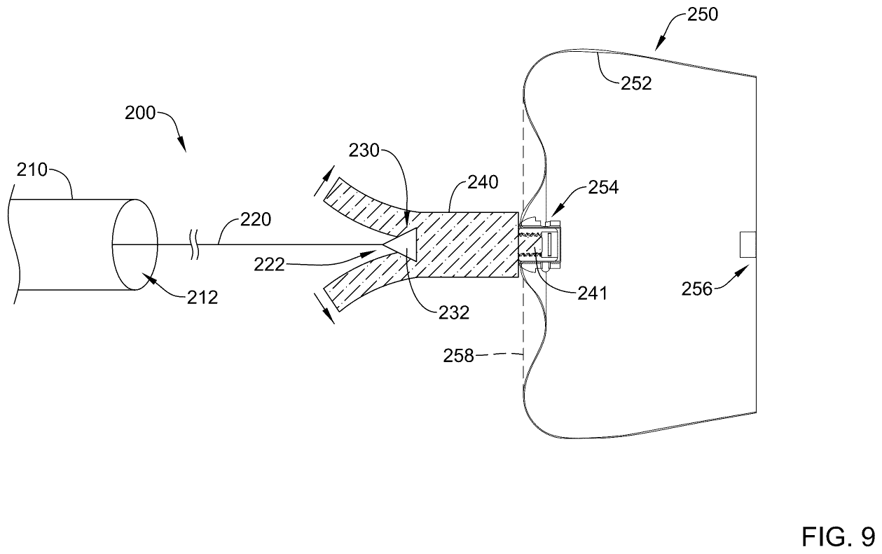

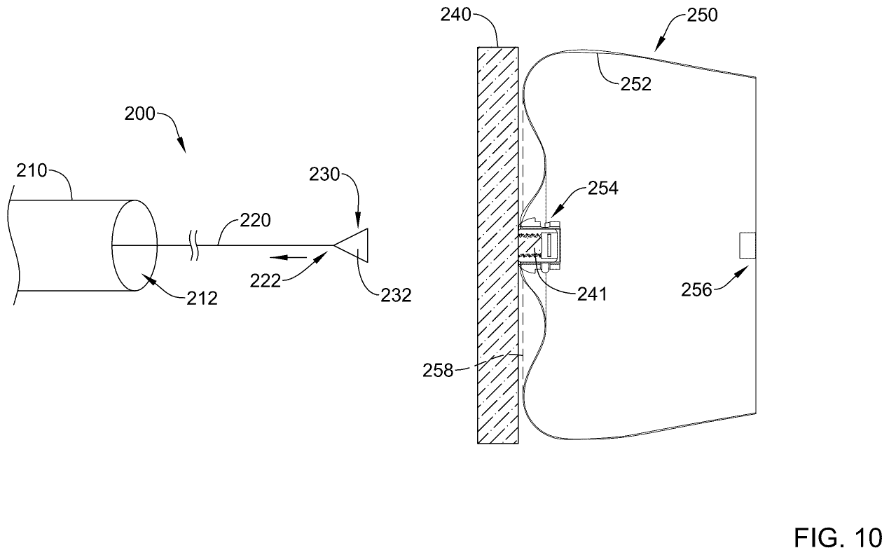

In some embodiments, the occlusive implant system 200 may comprise a medical implant 250 configured to occlude a left atrial appendage, as seen schematically in FIGS. 8-10. One non-limiting example of the medical implant 250 that is commercially available is the WATCHMAN™ from Boston Scientific. In some embodiments, the occlusive implant system 200 may also or alternatively be configured for use with other commercially available medical implants. The left atrial appendage is attached to and in fluid communication with the left atrium of the patient's heart. The left atrial appendage may have a complex geometry and/or irregular surface area. In some patients, the left atrial appendage may have a plurality of lobes and/or recesses extending in different directions.

The medical implant 250 may include an expandable framework 252 configured to shift between a collapsed configuration and a deployed configuration (e.g., FIGS. 8-10). In some embodiments, the expandable framework 252 may comprise a plurality of interconnected struts defining a plurality of cells. In some embodiments, the plurality of cells may be a plurality of closed cells. In some embodiments, the plurality of cells may be a plurality of open cells. In some embodiments, the plurality of cells may include a plurality of open cells and a plurality of closed cells in various combinations and/or arrangements. In some embodiments, the plurality of interconnected struts may converge, join, and/or connect at intersections or nodes.

The plurality of interconnected struts may be formed and/or cut from a tubular member. In some embodiments, the plurality of interconnected struts may be integrally formed and/or cut from a unitary member. In some embodiments, the plurality of interconnected struts may be integrally formed and/or cut from a unitary tubular member and subsequently formed and/or heat set to a desired shape in the deployed configuration. In some embodiments, the plurality of interconnected struts may be integrally formed and/or cut from a unitary flat member or sheet, and then rolled or formed into a tubular structure and subsequently formed and/or heat set to the desired shape in the deployed configuration. Some exemplary means and/or methods of making and/or forming the plurality of interconnected struts include laser cutting, machining, punching, stamping, electro discharge machining (EDM), chemical dissolution, etc. Other means and/or methods are also contemplated.

In some embodiments, the delivery catheter 210 may be configured to deliver the medical implant 250 to the left atrial appendage along with the piece of expandable foam 240. In some embodiments, the medical implant 250 and the piece of expandable foam 240 may be disposed within the lumen 212 of the delivery catheter 210 in the collapsed configuration and/or the delivery configuration. In some embodiments, the delivery catheter 210 may constrain the medical implant 250 and/or the expandable framework 252 in the collapsed configuration. In some embodiments, the medical implant 250 and/or the expandable framework 252 may be configured to shift from the collapsed configuration to the deployed configuration when the medical implant 250 is disposed distal of the distal opening of the lumen 212 and/or the delivery catheter 210, and/or when the medical implant 250 is unconstrained. In some embodiments, the medical implant 250 and/or the expandable framework 252 may be configured to shift from the collapsed configuration to the deployed configuration when the medical implant 250 is unconstrained by the delivery catheter 210. In at least some embodiments, the expandable framework 252 may be self-biased toward the deployed configuration.

In some embodiments, the piece of expandable foam 240 may be non-releasably coupled to the medical implant 250 and/or the expandable framework 252. In some embodiments, the piece of expandable foam 240 may be fixedly attached to the medical implant 250 and/or the expandable framework 252.

In some embodiments, the core wire 220 may be slidably and/or rotatably disposed within the lumen 212 of the delivery catheter 210. In some embodiments, the proximal end of the core wire 220 may extend proximally of a proximal end of the delivery catheter 210 and/or the proximal opening of the lumen 212 for manual manipulation by a clinician or practitioner. The core wire 220 may be configured to and/or may be capable of axially translating the medical implant 250 and the piece of expandable foam 240 relative to the delivery catheter 210. The delivery catheter 210 and/or the core wire 220 may have a selected level of axial stiffness and/or pushability characteristics while also having a selected level of flexibility to permit navigation through the patient's vasculature.

In some embodiments, the medical implant 250 and/or the expandable framework 252 may comprise a plurality of anchoring elements. In some embodiments, the plurality of anchoring elements may extend radially outward from the expandable framework 252 in the deployed configuration. In at least some embodiments, the plurality of anchoring elements may be configured to engage with tissue and/or may be configured to secure the medical implant 250 and/or the expandable framework 252 to tissue at a target site (e.g., the left atrial appendage, etc.). In some embodiments, the plurality of anchoring elements may be configured to prevent dislodgement and/or ejection of the medical implant 250 from the target site and/or the left atrial appendage.

In some embodiments, the medical implant 250 and/or the expandable framework 252 may comprise a proximal hub 254 and/or a distal hub 256. The plurality of interconnected struts may be joined together at and/or fixedly attached to the proximal hub 254 and/or the distal hub 256. In some embodiments, the proximal hub 254 and/or the distal hub 256 may be fixedly attached to the expandable framework 252 and/or the plurality of interconnected struts, such as by welding, adhesive bonding, brazing, soldering, etc.

In some embodiments, the piece of expandable foam 240 may be non-releasably coupled to the proximal hub 254. In some embodiments, the piece of expandable foam 240 may be fixedly attached to the proximal hub 254. In some embodiments, the piece of expandable foam 240 may comprise an implant connector portion 241 extending distally toward and/or into the proximal hub 254. In some embodiments, the implant connector portion 241 may be fixedly attached to the piece of expandable foam 240. In some embodiments, the implant connector portion 241 may be integrally formed with and/or monolithically formed with the piece of expandable foam 240. In some embodiments, the implant connector portion 241 may be configured to non-releasably engage and/or couple to the medical implant 250 and/or the proximal hub 254. In some embodiments, the implant connector portion 241 may be mechanically locked to medical implant 250 and/or the proximal hub 254. In some embodiments, the implant connector portion 241 may be adhesively bonded, welded, etc. to medical implant 250 and/or the proximal hub 254. Other configurations are also contemplated.

In some embodiments, the medical implant 250 may optionally include an occlusive covering 258 connected to, disposed on, disposed over, disposed about, and/or disposed radially outward of a proximal portion of the expandable framework 252 and/or the plurality of interconnected struts. In some embodiments, the occlusive covering 258 may be attached to the proximal hub 254 and/or may be attached to the expandable framework 252 at the proximal hub 254. In some embodiments, the occlusive covering 258 may extend radially outward from and/or may extend distally from the proximal hub 254. In some embodiments, the occlusive covering 258 may be attached and/or secured to the expandable framework 252 at a plurality of discrete locations. In some embodiments, one or more anchoring element(s) of the plurality of anchoring elements may extend through the occlusive covering 258. In some embodiments, the one or more anchoring element(s) of the plurality of anchoring elements extending through the occlusive covering 258 may attach and/or secure the occlusive covering 258 to the expandable framework 252.

In some embodiments, the occlusive covering 258 may include a membrane, a fabric, a mesh, a tissue element, or another suitable construction. In some embodiments, the occlusive covering 258 may be porous. In some embodiments, the occlusive covering 258 may be non-porous. In some embodiments, the occlusive covering 258 may be permeable or impermeable to blood and/or other fluids, such as water. In some embodiments, the occlusive covering 258 may be designed, sized, and/or configured to prevent thrombus and/or embolic material from passing out of the left atrial appendage into the left atrium and/or the patient's bloodstream. In some embodiments, the occlusive covering 258 (e.g., the membrane, the fabric, or the tissue element, etc.) promotes endothelization after implantation, thereby effectively and/or permanently removing the left atrial appendage from the patient's circulatory system. Some suitable, but non-limiting, examples of materials for the occlusive covering 258 are discussed below.

In some embodiments, the medical implant 250 may be configured, upon delivery and/or deployment within the left atrial appendage, to expand within and/or to engage with the left atrial appendage to seal off the left atrial appendage and/or substantially remove the left atrial appendage from the patient's circulatory system. The piece of expandable foam 240 and/or the occlusive disk may be configured, upon delivery to and/or deployment within the left atrial appendage, to expand within and/or to engage with the left atrial appendage to add additional sealing function to the medical implant 250 to further seal off the left atrial appendage and/or thereby remove the left atrial appendage from the patient's circulatory system.

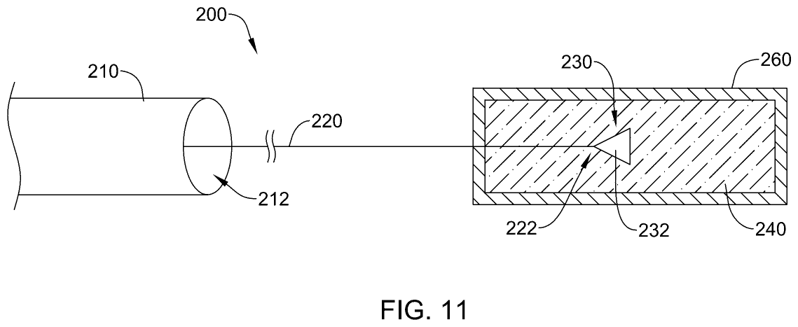

In some embodiments, the occlusive implant system 200 may comprise a shielding element 260 disposed on and/or about the piece of expandable foam 240 in the delivery configuration, as seen in FIG. 11. While illustrated and discussed in the context of the occlusive implant system 200, it shall be understood that the shielding element 260 may also be used in conjunction with the occlusive implant system 100 and the piece of expandable foam 140 associated therewith, and/or with respect to other examples and/or embodiments discussed herein/below. In some embodiments, the shielding element 260 may surround and/or encompass at least a portion of the piece of expandable foam 240. In some embodiments, the shielding element 260 may completely surround and/or completely encompass the piece of expandable foam 240.

In some embodiments, the shielding element 260 may be configured to degrade in vivo from exposure to fluid (e.g., water, blood, etc.) or another trigger condition. For example, the shielding element 260 may be configured to dissolve during, over, and/or after a predetermined period of time following deployment within the left atrial appendage.

In some embodiments, the shielding element 260 may be configured protect and/or shield the piece of expandable foam 240 from the trigger condition (e.g., a temperature change, an application or removal of an electrical current, a chemical change, exposure to fluid or a particular fluid type, etc.) that initiates or causes the piece of expandable foam 240 to shift from the delivery configuration toward and/or to the expanded configuration. In some embodiments, the shielding element 260 may constrain the piece of expandable foam 240 in the delivery configuration prior degradation of the shielding element 260 in vivo. In some embodiments, the shielding element 260 may be configured to delay the piece of expandable foam 240 shifting from the delivery configuration toward and/or to the expanded configuration. In some embodiments, the shielding element 260 may comprise a plurality of pores (not shown) formed therein. The plurality of pores may permit fluid to penetrate the shielding element 260 to interact with the piece of expandable foam 240 prior to degradation of the shielding element 260.

In some embodiments, the shielding element 260 may be formed as a movable covering disposed over and/or around the piece of expandable foam 240. In some embodiments, the shielding element 260 may be formed as a coating disposed on and/or around the piece of expandable foam 240. In some embodiments, the shielding element 260 may be formed as a shell disposed on and/or around the piece of expandable foam 240. In some embodiments, the shielding element 260 may comprise a naturally dissolving polymer, such as a simple sugar. In some embodiments, the shielding element 260 may comprise an accelerated dissolving polymer, such as a polymer that dissolves when exposed to an electrolyte or an electrical current, a polymer that dissolves when exposed to a temperature change (e.g., injection of hot water, etc.), a polymer that dissolves when exposed to a chemical change, a polymer that dissolves when exposed to water having a different salinity or saline concentration, etc. Other configurations are also contemplated.

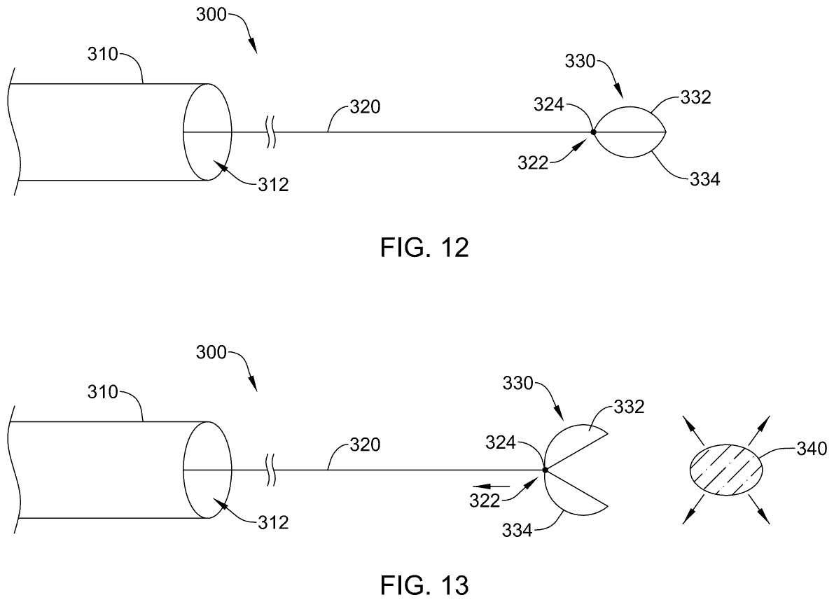

FIGS. 12-15 schematically illustrate selected aspects of an occlusive implant system 300. The occlusive implant system 300 may comprise a delivery catheter 310 having a lumen 312 extending therethrough. In some embodiments, the delivery catheter 310 may comprise a proximal hub and/or manifold (not shown). In some embodiments, the delivery catheter 310 may be formed from a tubular member. In some embodiments, the delivery catheter may comprise an additional lumen or lumens (e.g., a guidewire lumen, an aspiration lumen, a working lumen, etc.) extending therein and/or therethrough. Other configurations are also contemplated.

In some embodiments, the occlusive implant system 300 may comprise a core wire 320 slidably disposed within the delivery catheter 310 and/or the lumen 312 of the delivery catheter 310. In some embodiments, the core wire 320 may be formed with a solid cross-section. In some embodiments, the core wire 320 may be formed with a hollow cross-section (e.g., from a tubular member). In some embodiments, the core wire 320 may be formed from a metallic material. In some embodiments, the core wire 320 may be formed from a polymeric material. In some embodiments, the core wire 320 may be formed from a composite material.

The core wire 320 may comprise a clamshell enclosure 330 disposed at a distal end 322 of the core wire 320. In some embodiments, the clamshell enclosure 330 may be axially fixed to the distal end 322 of the core wire 320. In some embodiments, the clamshell enclosure 330 may be movably attached at the distal end 322 of the core wire 320. In some embodiments, the clamshell enclosure 330 may be pivotably attached to the distal end 322 of the core wire 320 by a hinge 324. In some embodiments, the hinge 324 may be fixedly attached to the distal end 322 of the core wire 320. In some embodiments, the hinge 324 may be adhesively bonded, welded, etc. to the distal end 322 of the core wire 320. Other configurations are also contemplated.

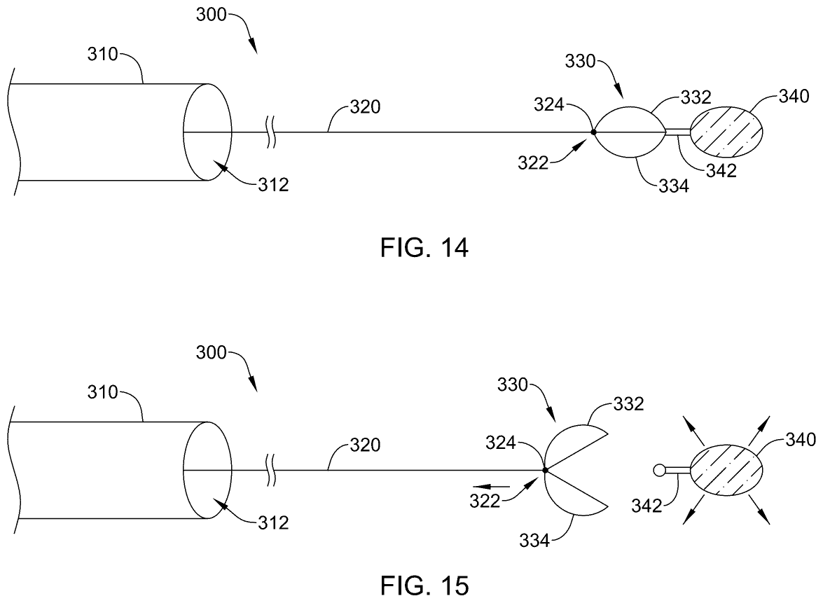

In some embodiments, the clamshell enclosure 330 may comprise a first shell portion 332 and a second shell portion 334. In some embodiments, the clamshell enclosure 330 may comprise additional shell portions. In some embodiments, the first shell portion 332 and the second shell portion 334 may be pivotably and/or hingedly coupled together at the distal end 322 of the core wire 320 and/or at the hinge 324. In some embodiments, the clamshell enclosure 330, and/or the first shell portion 332 and the second shell portion 334, may be configured to shift between a closed configuration (e.g., FIGS. 12, 14) and an open configuration (e.g., FIGS. 13, 15).

The occlusive implant system 300 may comprise a piece of expandable foam 340 configured to expand from a delivery configuration (e.g., a compressed configuration) to an expanded configuration. The clamshell enclosure 330 may be configured to deliver the piece of expandable foam 340 to a treatment site (e.g., the left atrial appendage) and thereafter release the piece of expandable foam 340 at and/or within the treatment site (e.g., the left atrial appendage).

In at least some embodiments, the piece of expandable foam 340 may be disposed within the clamshell enclosure 330 in the delivery configuration. Accordingly, in some embodiments, the piece of expandable foam 340 may be delivered to the treatment site within the clamshell enclosure 330 in the delivery configuration, and thereafter released from the clamshell enclosure 330 at and/or within the treatment site, where the piece of expandable foam 340 may shift from the delivery configuration toward and/or to the expanded configuration.

In some embodiments, the clamshell enclosure 330 may be releasably coupled to the piece of expandable foam 340 in the delivery configuration. In some embodiments, the piece of expandable foam 340 may comprise a marker element 342 disposed at a proximal end of the piece of expandable foam 340, as seen in FIGS. 14-15. In some embodiments, the marker element 342 may be fixedly attached to the proximal end of the piece of expandable foam 340. In some embodiments, the marker element 342 may be integrally formed with and/or monolithically formed with the piece of expandable foam 340. In some embodiments, the marker element 342 may be radiopaque. In some embodiments, the marker element 342 may comprise a portion of the piece of expandable foam 340 that has been doped with or coated with a radiopaque material. Other configurations are also contemplated.

In some embodiments, the clamshell enclosure 330 may be configured to releasably attach to the piece of expandable foam 340 and/or the marker element 342 to deliver the piece of expandable foam 340 to the treatment site, as seen in FIG. 14, and thereafter detach from the piece of expandable foam 340 and/or the marker element 342 to release the piece of expandable foam 340 at the treatment site, as seen in FIG. 15. Other configurations are also contemplated.

As discussed herein, the piece of expandable foam 340 may be configured to expand from the delivery configuration toward and/or to the expanded configuration in vivo (e.g., after deployment at the treatment site). While the piece of expandable foam 340 may be illustrated as having a particular shape, it shall be understood that the illustration is purely schematic and that other shapes and/or configurations are also contemplated within the scope of the disclosure. Additionally, it shall be understood that in some embodiments, the piece of expandable foam 340 may have a first shape in the delivery configuration and a second shape different from the first shape in the expanded configuration.

In some embodiments, the piece of expandable foam 340 may be self-biased toward the expanded configuration. In some embodiments, the piece of expandable foam 340 may comprise a shape memory polymer and/or a shape memory foam. The shape memory polymer and/or the shape memory foam may have multiple geometric and/or mechanical properties when exposed to temperature, moisture, and/or chemical environments, and/or changes therein. In some embodiments, the shape memory polymer and/or the shape memory foam may have a collapsibility ratio that is high. The collapsibility ratio is a ratio between an expanded size and a collapsed size. In some examples, the collapsibility ratio of the shape memory polymer and/or the shape memory foam may be at least 5 times, at least 7 times, at least 8 times, at least 9 times, at least 10 times, at least 12 times, or more. In one example, the piece of expandable foam 340 may have an outer diameter of about 32 millimeters in the expanded configuration and about 3 millimeters in the compressed configuration, producing a collapsibility ratio of at least 10 times (e.g., at least 10:1). Other configurations are also contemplated. In at least some embodiments, the piece of expandable foam 340 may be configured as open celled foam.

In some embodiments, the piece of expandable foam 340 may be configured to shift from the delivery configuration toward and/or to the expanded configuration after being exposed and/or subjected to a trigger condition (e.g., a temperature change, an application or removal of an electrical current, a chemical change, exposure to fluid or a particular fluid type, etc.).

The piece of expandable foam 340 and/or the shape memory foam may be formed from a biocompatible material. In at least some embodiments, the piece of expandable foam 340 may be non-biodegradable and/or non-bioabsorbable. In some alternative embodiments, the piece of expandable foam 340 may be biodegradable and/or bioabsorbable over time. In some embodiments, the piece of expandable foam 340 may be configured to prevent thrombus formation. In some embodiments, the piece of expandable foam 340 may include anti-thrombus medicament(s). In some embodiments, the piece of expandable foam 340 may be configured to absorb blood and/or bodily fluid(s). In some embodiments, the piece of expandable foam 340 may be configured to trap thrombus. In some embodiments, the piece of expandable foam 340 may be configured to promote endothelization and/or tissue ingrowth. In some embodiments, the piece of expandable foam 340 may include a coating, a material, and/or a component that promotes endothelization and/or tissue ingrowth. Other configurations are also contemplated.

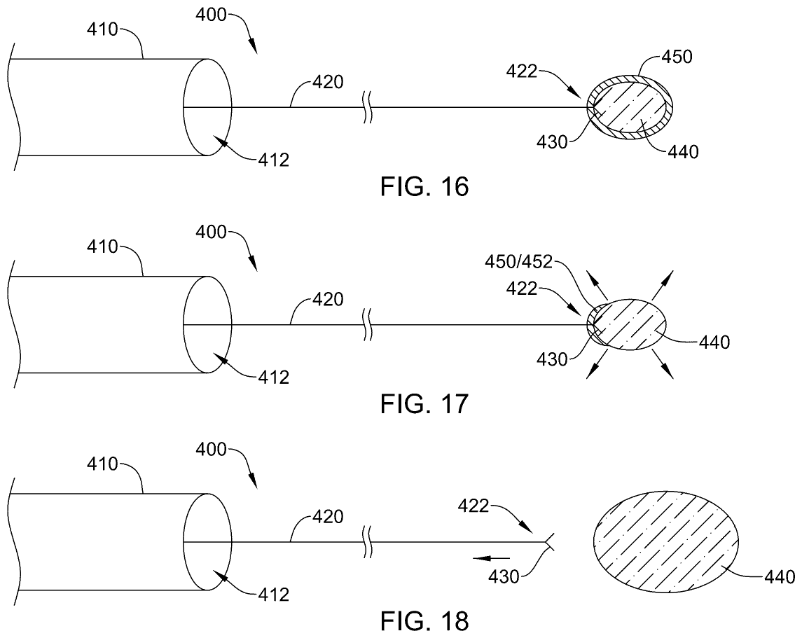

FIGS. 16-18 schematically illustrate selected aspects of an occlusive implant system 400. The occlusive implant system 400 may comprise a delivery catheter 410 having a lumen 412 extending therethrough. In some embodiments, the delivery catheter 410 may comprise a proximal hub and/or manifold (not shown). In some embodiments, the delivery catheter 410 may be formed from a tubular member. In some embodiments, the delivery catheter may comprise an additional lumen or lumens (e.g., a guidewire lumen, an aspiration lumen, a working lumen, etc.) extending therein and/or therethrough. Other configurations are also contemplated.

In some embodiments, the occlusive implant system 400 may comprise a core wire 420 slidably disposed within the delivery catheter 410 and/or the lumen 412 of the delivery catheter 410. In some embodiments, the core wire 420 may be formed with a solid cross-section. In some embodiments, the core wire 420 may be formed with a hollow cross-section (e.g., from a tubular member). In some embodiments, the core wire 420 may be formed from a metallic material. In some embodiments, the core wire 420 may be formed from a polymeric material. In some embodiments, the core wire 420 may be formed from a composite material.

The core wire 420 may comprise a connection element 430 disposed at a distal end 422 of the core wire 420. In at least some embodiments, the connection element 430 may be fixedly attached to the distal end 422 of the core wire 420. In some embodiments, the connection element 430 may be adhesively bonded, etc. to the distal end 422 of the core wire 420. In some embodiments, the connection element 430 may be integrally formed with the core wire 420. In some embodiments, the connection element 430 may be monolithically formed with the core wire 420. Other configurations are also contemplated.

The occlusive implant system 400 may comprise a piece of expandable foam 440 coupled to the connection element 430. The piece of expandable foam 440 may be configured to expand from a delivery configuration (e.g., a compressed configuration) to an expanded configuration. While the piece of expandable foam 440 may be illustrated as having a particular shape, it shall be understood that the illustration is purely schematic and that other shapes and/or configurations are also contemplated within the scope of the disclosure. Additionally, it shall be understood that in some embodiments, the piece of expandable foam 440 may have a first shape in the delivery configuration and a second shape different from the first shape in the expanded configuration.

In some embodiments, the piece of expandable foam 440 may be coupled to the connection element 430 initially while the piece of expandable foam 440 is in the expanded configuration. Accordingly, in some embodiments, a portion of the connection element 430 may be disposed within the piece of expandable foam 440. Thereafter, the piece of expandable foam 440 may be compressed toward and/or to the delivery configuration such that the piece of expandable foam 440 is compressed onto the portion of the connection element 430 disposed within the piece of expandable foam 440 in the delivery configuration. In some embodiments, the connection element 430 may be disposed entirely within the piece of expandable foam 440 in the delivery configuration. In some embodiments, the connection element 430 may be prevented from disengaging from the piece of expandable foam 440 when the piece of expandable foam 440 is in the delivery configuration.

As discussed herein, the piece of expandable foam 440 may be configured to expand from the delivery configuration toward and/or to the expanded configuration in vivo (e.g., after deployment at the treatment site). In some embodiments, the piece of expandable foam 440 may be self-biased toward the expanded configuration. In some embodiments, the piece of expandable foam 440 may comprise a shape memory polymer and/or a shape memory foam. The shape memory polymer and/or the shape memory foam may have multiple geometric and/or mechanical properties when exposed to temperature, moisture, and/or chemical environments, and/or changes therein. In some embodiments, the shape memory polymer and/or the shape memory foam may have a collapsibility ratio that is high. The collapsibility ratio is a ratio between an expanded size and a collapsed size. In some examples, the collapsibility ratio of the shape memory polymer and/or the shape memory foam may be at least 5 times, at least 7 times, at least 8 times, at least 9 times, at least 10 times, at least 12 times, or more. In one example, the piece of expandable foam 440 may have an outer diameter of about 32 millimeters in the expanded configuration and about 3 millimeters in the compressed configuration, producing a collapsibility ratio of at least 10 times (e.g., at least 10:1). Other configurations are also contemplated. In at least some embodiments, the piece of expandable foam 440 may be configured as open celled foam.

In some embodiments, the piece of expandable foam 440 may be configured to shift from the delivery configuration toward and/or to the expanded configuration after being exposed and/or subjected to a trigger condition (e.g., a temperature change, an application or removal of an electrical current, a chemical change, etc.).

The piece of expandable foam 440 and/or the shape memory foam may be formed from a biocompatible material. In at least some embodiments, the piece of expandable foam 440 may be non-biodegradable and/or non-bioabsorbable. In some alternative embodiments, the piece of expandable foam 440 may be biodegradable and/or bioabsorbable over time. In some embodiments, the piece of expandable foam 440 may be configured to prevent thrombus formation. In some embodiments, the piece of expandable foam 440 may include anti-thrombus medicament(s). In some embodiments, the piece of expandable foam 440 may be configured to absorb blood and/or bodily fluid(s). In some embodiments, the piece of expandable foam 440 may be configured to trap thrombus. In some embodiments, the piece of expandable foam 440 may be configured to promote endothelization and/or tissue ingrowth. In some embodiments, the piece of expandable foam 440 may include a coating, a material, and/or a component that promotes endothelization and/or tissue ingrowth. Other configurations are also contemplated.

In some embodiments, the piece of expandable foam 440 may have a generally open structure (e.g., the piece of expandable foam 440 may have pores or open cells) in the expanded configuration. In some embodiments, the piece of expandable foam 440 may be configured to receive and/or accept the connection element 430 therein in the expanded configuration. Upon compression and/or crimping of the piece of expandable foam 440 onto the connection element 430, and/or toward and/or to the delivery configuration, the connection element 430 is or would be unable to disengage from, and/or is or would be prevented from, disengaging from the piece of expandable foam 440.

In some embodiments, the connection element 430 may comprise and/or may be a T-shape. In some embodiments, the connection element 430 may be and/or may have a three-dimensional shape. In some embodiments, the three-dimensional shape may include a narrow end and a wide end, wherein the narrow end is configured to couple directly to the core wire 420 and the wide end is disposed opposite the narrow end and/or is spaced apart from the narrow end. As such, in some embodiments, the connection element 430 may have a pyramidal shape, a prismatic shape, a conical shape, etc. In some embodiments, the connection element 430 may be fixedly attached to the distal end 422 of the core wire 420. In some embodiments, the narrow end of the connection element 430 may be fixedly attached directly to the distal end 422 of the core wire 420. In some embodiments, the connection element 430 may be integrally formed with the core wire 420 and/or monolithically formed with the core wire 420 with the wide end of the connection element 430 disposed opposite and/or spaced apart from the distal end 422 of the core wire 420. Other configurations are also contemplated.

In some embodiments, a portion of the connection element 430, and/or an entirety of the connection element 430, may be inserted into the piece of expandable foam 440 and/or the generally open structure of the piece of expandable foam 440 in the expanded configuration. In some embodiments, the piece of expandable foam 440 may be formed around the portion of the connection element 430, and/or the entirety of the connection element 430. Thereafter, the piece of expandable foam 440 may be compressed and/or crimped onto the portion of the connection element 430 that is disposed within the piece of expandable foam 440. Once the piece of expandable foam 440 has been compressed and/or crimped onto the portion of the connection element 430 that is disposed within the piece of expandable foam 440, the connection element 430 may be prevented from disengaging from and/or pulling out of the piece of expandable foam 440.

In some embodiments, a portion of the connection element 430, and/or an entirety of the connection element 430, may be engaged with and/or pressed against an outer surface of the piece of expandable foam 440. In some embodiments, a first portion of the connection element 430 may be engaged with and/or pressed against an outer surface of the piece of expandable foam 440, and a second portion of the connection element 430 may be inserted into the piece of expandable foam 440 and/or the generally open structure of the piece of expandable foam 440. Other configurations are also contemplated.

In some embodiments, the occlusive implant system 400 may comprise a polymer coating 450 disposed on and/or around the piece of expandable foam 440 and/or disposed on and/or around the outer surface of the piece of expandable foam 440. In some embodiments, the polymer coating 450 may be disposed outward of the connection element 430 with respect to the piece of expandable foam 440, thereby coupling and/or securing the connection element 430 to the piece of expandable foam 440. In some embodiments, the polymer coating 450 may surround and/or encompass at least a portion of the piece of expandable foam 440. In some embodiments, the polymer coating 450 may completely surround and/or completely encompass the piece of expandable foam 440.

In some embodiments, the polymer coating 450 may be configured to degrade in vivo to release the piece of expandable foam 440 from the connection element 430, as seen in FIG. 18. In some embodiments, the polymer coating 450 may be configured to degrade in vivo from exposure to fluid (e.g., water, blood, etc.) or another trigger condition. For example, the polymer coating 450 may be configured to dissolve during, over, and/or after a predetermined period of time following deployment within the left atrial appendage.

In some embodiments, the polymer coating 450 may be configured to protect and/or shield the piece of expandable foam 440 from the trigger condition (e.g., a temperature change, an application or removal of an electrical current, a chemical change, exposure to fluid or a particular fluid type, etc.) that initiates or causes the piece of expandable foam 440 to shift from the delivery configuration toward and/or to the expanded configuration. In some embodiments, the polymer coating 450 may constrain the piece of expandable foam 440 in the delivery configuration prior to degradation of the polymer coating 450 in vivo. In some embodiments, the polymer coating 450 may be configured to delay the piece of expandable foam 440 shifting from the delivery configuration toward and/or to the expanded configuration. In some embodiments, the polymer coating 450 may comprise a plurality of pores (not shown) formed therein. The plurality of pores may permit fluid to penetrate the polymer coating 450 to interact with the piece of expandable foam 440 prior to degradation of the polymer coating 450.

In some embodiments, the polymer coating 450 may comprise a naturally dissolving polymer, such as a simple sugar. In some embodiments, the polymer coating 450 may comprise an accelerated dissolving polymer, such as a polymer that dissolves when exposed to an electrolyte or an electrical current, a polymer that dissolves when exposed to a temperature change (e.g., injection of hot water, etc.), a polymer that dissolves when exposed to a chemical change, a polymer that dissolves when exposed to water having a different salinity or saline concentration, etc. Other configurations are also contemplated.

In some embodiments, the polymer coating 450 may be configured to degrade in stages. For example, upon deployment at the treatment site (e.g., within the left atrial appendage), a distal portion of the polymer coating 450 may be configured to degrade before a proximal portion 452 of the polymer coating 450. In some embodiments, the proximal portion 452 of the polymer coating 450 may be thicker than the distal portion of the polymer coating 450, such that the proximal portion 452 requires more time to degrade. In some embodiments, the polymer coating 450 may be applied in a thinning gradient from the proximal portion 452 of the polymer coating 450 to the distal portion of the polymer coating 450. In some embodiments, the proximal portion 452 of the polymer coating 450 may have a different chemical composition than the distal portion of the polymer coating 450. Other configurations are also contemplated. The proximal portion 452 of the polymer coating 450 may be disposed over the connection element 430. As such, the proximal portion 452 of the polymer coating 450 may be configured to couple and/or attach the piece of expandable foam 440 to the connection element 430 as the distal portion of the polymer coating 450 degrades. In some embodiments, the proximal portion 452 of the polymer coating 450 may have time-delayed degradation characteristics.

In some embodiments, the piece of expandable foam 440 or at least a portion of the piece of expandable foam 440 may be configured to begin expanding from the delivery configuration toward and/or to the expanded configuration before the polymer coating 450 releases the piece of expandable foam 440 from the connection element 430. In some embodiments, the piece of expandable foam 440 or at least a portion of the piece of expandable foam 440 (e.g., a distal portion of the piece of expandable foam 440) may be configured to begin expanding from the delivery configuration toward and/or to the expanded configuration before the proximal portion 452 of the polymer coating 450 releases the piece of expandable foam 440 from the connection element 430. In some embodiments, the piece of expandable foam 440 or at least a portion of the piece of expandable foam 440 (e.g., a distal portion of the piece of expandable foam 440) may be configured to begin expanding from the delivery configuration toward and/or to the expanded configuration after the distal portion of the polymer coating 450 degrades and before the proximal portion 452 of the polymer coating 450 degrades and/or releases the piece of expandable foam 440 from the connection element 430, as seen in FIG. 17. Other configurations are also contemplated.

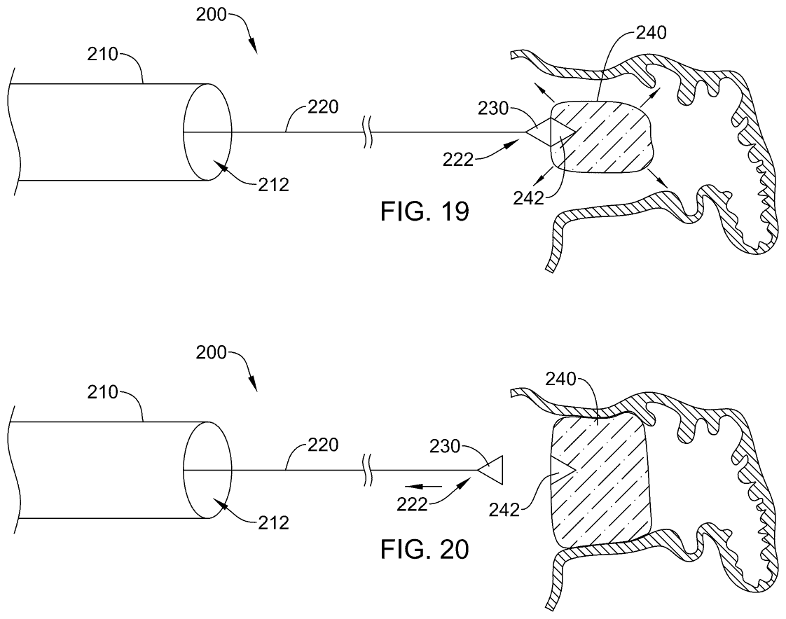

FIGS. 19-23 schematically illustrates selected aspects of the occlusive implant system 200 disclosed herein. In addition or alternatively to the aspects and/or configurations disclosed above, in some embodiments, the piece of expandable foam 240 may comprise a second connection element 242. The second connection element 242 may be configured to engage with the connection element 230. In some embodiments, the second connection element 242 may be selectively coupled to the connection element 230. In some embodiments, the second connection element 242 may be releasably coupled to the connection element 230. In at least some embodiments, the second connection element 242 may be fixedly attached and/or permanently attached to the piece of expandable foam 240. In some embodiments, the second connection element 242 may be adhesively bonded to the piece of expandable foam 240. In some embodiments, the second connection element 242 may be mechanically secured to the piece of expandable foam 240 such that the second connection element 242 is prevented from detaching and/or decoupling from the piece of expandable foam 240.

In some embodiments, the second connection element 242 may be at least partially disposed within the piece of expandable foam 240 in the delivery configuration. In some embodiments, the second connection element 242 may be disposed completely within the piece of expandable foam 240 in the delivery configuration. In some embodiments, the piece of expandable foam 240 may provide and/or add structural support to the second connection element 242 and/or to the engagement between the connection element 230 and the second connection element 242. In some embodiments, the second connection element 242 may be at least partially disposed within the piece of expandable foam 240 in the delivery configuration and the expanded configuration. In some embodiments, the second connection element 242 may be disposed completely within the piece of expandable foam 240 in the delivery configuration and the expanded configuration. Other configurations are also contemplated.

In at least some embodiments, the second connection element 242 may be configured to selectively disengage and/or selectively detach from the connection element 230 in vivo after the piece of expandable foam 240 has been deployed at the treatment site (e.g., within the left atrial appendage). In some embodiments, tension applied to the connection element 230 after the piece of expandable foam 240 has expanded to the expanded configuration causes the connection element 230 to disengage from the second connection element 242. In at least some embodiments, the second connection element 242 may be configured to remain with and/or in the piece of expandable foam 240 after the connection element 230 is disengaged from the second connection element 242.

In some embodiments, a magnetic force may selectively couple the connection element 230 to the second connection element 242. In one example, the connection element 230 and/or the second connection element 242 may be a permanent magnet. In some embodiments, whichever of the connection element 230 and the second connection element 242 is not a permanent magnet may be constructed of a magnetic material and/or a material doped with magnetic particles such that it is magnetically attracted to the permanent magnet. In some alternative embodiments, the piece of expandable foam 240 may be the second connection element 242 itself. For example, the piece of expandable foam 240 may be constructed of a magnetic material and/or a material doped with magnetic particles such that it is magnetically attracted to the permanent magnet. After the piece of expandable foam 240 has been deployed (e.g., FIG. 19) and has expanded to the expanded configuration (e.g., FIG. 20), tension may be applied to the core wire 220 and/or the connection element 230 to decouple and/or detach the connection element 230 from the second connection element 242 in vivo.

In some embodiments, the magnetic force may be greater when the piece of expandable foam 240 is in the delivery configuration than when the piece of expandable foam 240 is in the expanded configuration. When the piece of expandable foam 240 is in the delivery configuration, tension applied to the core wire 220 and/or the connection element 230 may be unable to overcome the magnetic force to decouple and/or detach the connection element 230 from the second connection element 242. After the piece of expandable foam 240 has expanded to the expanded configuration, the magnetic force may be reduced such that tension applied to the core wire 220 and/or the connection element 230 may be able to overcome the magnetic force to decouple and/or detach the connection element 230 from the second connection element 242. Other configurations are also contemplated.

In another example, the connection element 230 may be an electromagnet. The core wire 220 may comprise an electrical pathway coupled to the connection element 230. The electromagnet may be activated prior to delivery of the piece of expandable foam 240 to the treatment site (e.g., within the left atrial appendage). While the electromagnet is active, tension applied to the core wire 220 and/or the connection element 230 may be unable to overcome the magnetic force to decouple and/or detach the connection element 230 from the second connection element 242. After the piece of expandable foam 240 has been deployed (e.g., FIG. 19) and has expanded to the expanded configuration (e.g., FIG. 20), the electromagnet may be deactivated to decouple and/or detach the connection element 230 from the second connection element 242 in vivo. Other configurations are also contemplated.

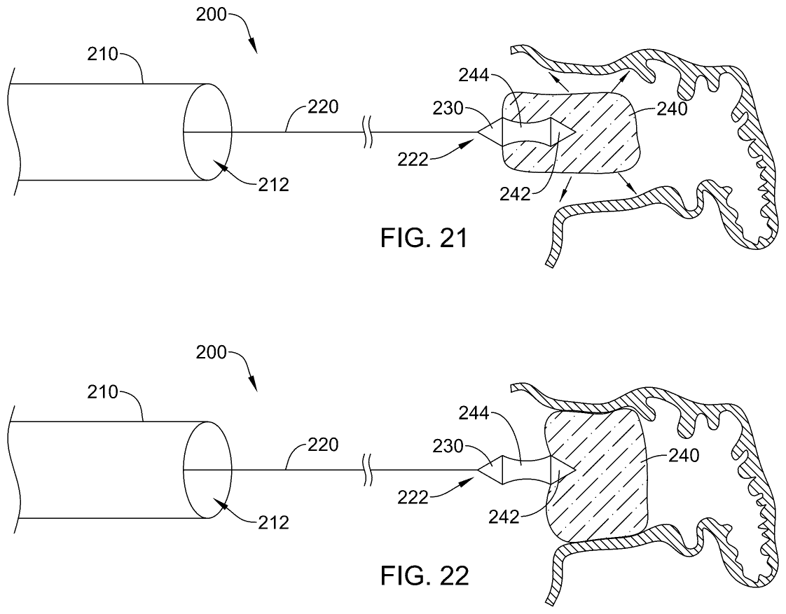

In some embodiments, the occlusive implant system 200, the connection element 230, and/or the second connection element 242 may further comprise a third connection element 244, as seen in FIGS. 21-23. In some embodiments, the connection element 230 may be fixedly attached to the second connection element 242 by the third connection element 244.

In some embodiments, the second connection element 242 and/or the third connection element 244 may be at least partially disposed within the piece of expandable foam 240 in the delivery configuration. In some embodiments, the second connection element 242 and/or the third connection element 244 may be disposed completely within the piece of expandable foam 240 in the delivery configuration. In some embodiments, the piece of expandable foam 240 may provide and/or add structural support to the second connection element 242 and/or the third connection element 244, and/or to the engagement between the connection element 230, the second connection element 242, and/or the third connection element 244. In some embodiments, the second connection element 242 and/or the third connection element 244 may be at least partially disposed within the piece of expandable foam 240 in the delivery configuration and the expanded configuration. In some embodiments, the second connection element 242 and/or the third connection element 244 may be disposed completely within the piece of expandable foam 240 in the delivery configuration and the expanded configuration. Other configurations are also contemplated.

In at least some embodiments, the second connection element 242 and/or the third connection element 244 may be configured to selectively disengage and/or selectively detach from the connection element 230 in vivo after the piece of expandable foam 240 has been deployed at the treatment site (e.g., within the left atrial appendage). In some embodiments, tension applied to the connection element 230 after the piece of expandable foam 240 has expanded to the expanded configuration causes the connection element 230, the second connection element 242, and/or the third connection element 244 to disengage the connection element 230 from the second connection element 242. In some embodiments, tension applied to the connection element 230 after the piece of expandable foam 240 has expanded to the expanded configuration causes the connection element 230, the second connection element 242, and/or the third connection element 244 to break to disengage the connection element 230 from the second connection element 242.

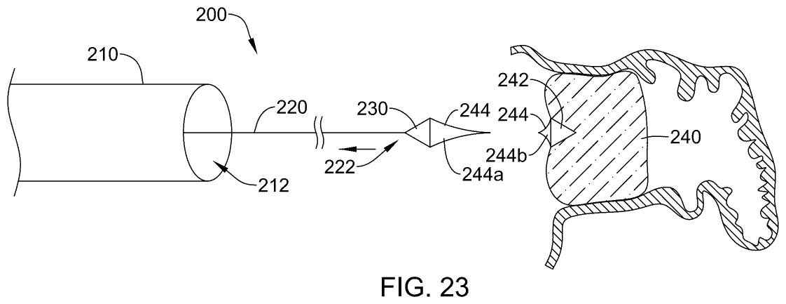

In some embodiments, the third connection element 244 may be integrally formed with the connection element 230 and/or the second connection element 242. In some embodiments, the third connection element 244 may be monolithically formed with the connection element 230 and/or the second connection element 242. In some embodiments, the third connection element 244 may be designed and configured with characteristics that permit and/or promote failure of the third connection element 244 under preselected conditions. In some embodiments, the preselected conditions may include a minimum amount of tension. The minimum amount of tension may be unachievable prior to expansion of the piece of expandable foam 240. That is, merely pulling on the core wire 220 before the piece of expandable foam 240 has expanded to the expanded configuration (e.g., in free space, when not engaged with the left atrial appendage, etc.) may be unable to apply sufficient tensile force to the third connection element 244 to cause failure of the third connection element 244. However, after the piece of expandable foam 240 has been deployed (e.g., FIG. 21) and has expanded to the expanded configuration (e.g., FIG. 22), tension applied to the core wire 220 and/or the connection element 230 may transfer tensile force to the third connection element 244, thereby causing stretching of the third connection element 244 and eventually failure of the third connection element 244, as seen in FIG. 23. Accordingly, tension applied to the connection element 230 after the piece of expandable foam 240 has expanded to the expanded configuration within the left atrial appendage causes the connection element 230 to disengage from the second connection element 242.

In some embodiments, tensile failure of the third connection element 244 may cause a proximal portion 244A of the third connection element 244 and the connection element 230 fixedly attached thereto to disengage from a distal portion 244B of the third connection element 244 and the second connection element 242 fixedly attached thereto. The distal portion 244B of the third connection element 244 and the second connection element 242 may be configured to remain coupled to the piece of expandable foam 240 and/or to remain within the treatment site (e.g., the left atrial appendage) with the piece of expandable foam 240 while the proximal portion 244A of the third connection element 244 is removed along with the core wire 220 and the connection element 230. Other configurations are also contemplated.

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the delivery catheter, the core wire, the piece of expandable foam, the clamshell enclosure, the medical implant, the expandable framework, the occlusive element, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An occlusive implant system, comprising:
a delivery catheter having a lumen extending therethrough;
a core wire slidably disposed within the delivery catheter, wherein the core wire comprises a connection element disposed at a distal end of the core wire; and
a piece of foam coupled to the connection element, the piece of foam being configured to expand from a delivery configuration to an expanded configuration.

2. The occlusive implant system of claim 1, wherein a portion of the connection element is disposed within the piece of foam in the delivery configuration.

3. The occlusive implant system of claim 2, wherein the piece of foam is compressed onto the portion of the connection element disposed within the piece of foam in the delivery configuration such that the connection element is prevented from disengaging from the piece of foam when the piece of foam is in the delivery configuration.

4. The occlusive implant system of claim 2, wherein the connection element is a helical coil.

5. The occlusive implant system of claim 4, wherein the connection element is configured to be unscrewed from the piece of foam when the piece of foam is in the expanded configuration.

6. The occlusive implant system of claim 4, wherein the connection element is configured to straighten and pull out of the piece of foam when tension is applied to the connection element and the piece of foam is in the expanded configuration.

7. The occlusive implant system of claim 2, wherein the connection element is a wedge.

8. The occlusive implant system of claim 7, wherein the connection element is disposed entirely within the piece of foam in the delivery configuration.

9. The occlusive implant system of claim 7, wherein expansion of the piece of foam to the expanded configuration releases the connection element from the piece of foam.

10. The occlusive implant system of claim 7, further comprising a shielding element disposed about the piece of foam in the delivery configuration.

11. The occlusive implant system of claim 7, wherein the piece of foam is non-releasably coupled to a medical implant configured to occlude a left atrial appendage, the medical implant comprising an expandable framework.

12. The occlusive implant system of claim 11, wherein the piece of foam forms an occlusive disk in the expanded configuration.

13. The occlusive implant system of claim 1, wherein the piece of foam comprises a second connection element disposed therein, the second connection element being configured to selectively disengage from the connection element to release the piece of foam.

14. The occlusive implant system of claim 13, wherein a magnetic force selectively couples the connection element to the second connection element.

15. The occlusive implant system of claim 13, wherein tension applied to the connection element after the piece of foam has expanded to the expanded configuration causes the connection element to disengage from the second connection element.

16. An occlusive implant system, comprising:
a delivery catheter having a lumen extending therethrough;
a core wire slidably disposed within the delivery catheter, wherein the core wire comprises a clamshell enclosure disposed at a distal end of the core wire; and
a piece of foam configured to expand from a delivery configuration to an expanded configuration;
wherein the clamshell enclosure is configured to deliver the piece of foam to a treatment site and thereafter release the piece of foam at the treatment site.

17. The occlusive implant system of claim 16, wherein the piece of foam is disposed within the clamshell enclosure in the delivery configuration.

18. The occlusive implant system of claim 16, wherein the clamshell enclosure is releasably coupled to the piece of foam in the delivery configuration.

19. The occlusive implant system of claim 18, wherein the piece of foam comprises a marker element disposed at a proximal end of the piece of foam, and the clamshell enclosure is configured to releasably attach to the marker element to deliver the piece of foam to a treatment site and thereafter detach from the marker element to release the piece of foam at the treatment site.

20. An occlusive implant system, comprising:
a delivery catheter having a lumen extending therethrough;
a core wire slidably disposed within the delivery catheter, wherein the core wire comprises a connection element disposed at a distal end of the core wire;
a piece of foam engaged with the connection element, the piece of foam being configured to expand from a delivery configuration to an expanded configuration; and
a polymer coating disposed on the piece of foam in the delivery configuration, wherein the polymer coating is configured to degrade in vivo to release the piece of foam from the connection element;
wherein the piece of foam is configured to begin expanding from the delivery configuration to the expanded configuration before the polymer coating releases the piece of foam from the connection element.
